(19) United States
(12) Patent Application Publication
(10) Pub. No.: US 2010/0061885 A1
(43) Pub. Date: Mar. 11, 2010

(54) INSTRUMENT FOR DETERMINING OZONE CONCENTRATION

(76) Inventor: Phillip E. Harley, Hexham (GB)

Correspondence Address: MACMILLAN SOBANSKI & TODD, LLC, ONE MARITIME PLAZA FIFTH FLOOR, 720 WATER STREET, TOLEDO, OH, 43604-1619, US

(21) Appl. No.: 12/555,820

(22) Filed: Sep. 9, 2009

(30) Foreign Application Priority Data
Sep. 9, 2008 (GB) 0816445.1

(51) Int. Cl. A61L 2/24; B01D 46/00; B01D 46/46

(52) U.S. Cl. 422/3; 96/397; 95/1

(57) ABSTRACT

An instrument for determining ozone concentration in a gaseous fluid includes a chamber, at least one filter adapted to remove particulates from gaseous fluid entering the chamber, and at least one filter adapted to remove ozone from gaseous fluid entering the chamber. At least one element is arranged to draw gaseous fluid into the chamber. An ultra-violet source located at one end of the chamber and configured to generate a substantially collimated beam of radiation having a wave length in the range 240 to 290 nm, and an ultra-violet sensor is arranged at the other end of the chamber and configured to receive the ultra-violet light emitted by the said ultra-violet source.

### BACKGROUND OF THE INVENTION

The invention relates to the determination of ozone concentration in an atmosphere, and in particular to an instrument for determining ozone concentration in an atmosphere and a method of using such an instrument.

Ozone is present in the atmosphere at all times. Excess ozone can act as a pollutant. Ozone also has uses in a number of fields. Where ozone is deployed in an environment there is usually a need to monitor the concentration of ozone present.

There is a need to determine ozone concentrations in a number of different settings. For example, there is a limit for ozone concentrations in environments occupied by persons for health and safety purposes. If the ozone concentration exceeds certain thresholds then personnel must be removed from the environment.

Some crop stores and mobile transport containers use environments modified with ozone to inhibit microbial decay. The concentration of ozone in the store or transport container must be monitored and maintained between defined limits.

Ozone concentration must also be determined where ozone is used as a sterilising agent. In order to be certain that an object has been sterilised it is necessary to record a guaranteed minimum concentration as having been delivered. The concentration of ozone as an atmospheric pollutant is also routinely measured.

There are three primary classes of instrument for detecting and measuring ozone concentrations in air. The first takes advantage of the optical absorption of light passing through a sample of air under investigation. It is well known that ozone strongly absorbs light in the short wavelength ultra-violet region of the spectrum, commonly referred to as UV-C radiation. By positioning a source of UV-C radiation at a known distance from a detector of the radiation, then the well known Beer-Lambert law may be applied to calculate the expected loss in transmission between the source and the detector, hence revealing the average concentration of ozone present between them. Such instruments exhibit a number of practical problems that limit their usefulness. Until very recently there are few light sources available that offer the combination of a desirable emission wavelength, and stability in output, whilst representing a sufficiently small point source to allow efficient beam optics to be established. The universal choice of light source to date has been the mercury discharge lamp. Whilst one of its narrow emission lines at 254 nm is ideal for detecting ozone, it is bulky; has an extended emitting area (i.e. it is far from being the ideal point source necessary for good beam formation); requires a high voltage power supply; is inefficient; has a very limited life (typically only several months of continuous operation); and it contains mercury; (a listed substance under the European Directive on the Restrictions of the Use of Certain Hazardous Substances in Electrical and Electronic Equipment—RoHS). Furthermore, broad emission from the mercury lamp extends into the visible part of the spectrum and so expensive UV transmitting, visible light blocking filters have to be employed in front of the detector device to prevent its interference with the measurement. Also, to further prevent the effects of ambient light entering the measurement device, current UV-C ozone measuring instruments utilise an enclosed tube through which the sampled air is pulled by means of a vacuum pump (usually a reciprocating diaphragm pump). Apart from the limited continuous duty operational life of such a pump, and its power demands, there is evidence that deposits on the walls of the tube can result in errors in the calculated Beer-Lambert response of the instrument, resulting in unreliable sensitivity, and so requiring frequent recalibration. Finally it should be noted that the mercury discharge lamp may not easily be modulated or have its output chopped, thus preventing it being used in systems that make use of such modulation by means of electrical filtering, or the powerful technique of phase sensitive detection that those skilled in the art apply to the recovery of small electrical signals.

A second class of instruments makes use of certain special semiconductor devices to sense ozone in an air sample passed across them. A particularly good example is a device manufactured by City Technology Group which utilises a mixed metal oxide semiconductor material that exhibits an impedance change when its surface is in contact with ozone. Other manufacturers offer semiconductor sensors based on tin-oxide that behave in a similar way. All the gas sensing semiconductor devices require complex electronics to compensate for their non-linear and widely varying behaviour. A major obstacle to their application is their cross sensitivity to other gases and volatiles present in the atmosphere. Traces of volatile organic compounds present in the sampled air can interfere with the sensor rendering it completely ineffective with potentially dangerous consequences to persons present in the environment. It is known that the function of semiconductor sensors when used to control ozone in harvested crop stores can be badly affected by the presence of various organics including turpenes found in association with citrus fruits and other crops.

A third class of sensors makes use of electrochemical effects whereby ozone permeates a membrane and is absorbed by an electrolyte in an electrochemical cell, resulting in a detectable change in the cell's electrical characteristics. These sensors, whilst robust, are insensitive to the levels of ozone of interest to health and safety professionals, and similar low ozone concentration applications.

It would therefore be desirable to provide an improved instrument for detecting ozone.

It would also be desirable to provide improved ozone sterilisation equipment.

It would also be desirable to provide an improved crop store.

It would also be desirable to provide an improved mobile transport container.

### SUMMARY OF THE INVENTION

According a first aspect of the invention there is provided an instrument for determining ozone concentration in a gaseous fluid comprising a chamber, at least one filter adapted to remove particulates from gaseous fluid entering the chamber, and at least one filter adapted to remove ozone from gaseous fluid entering the chamber, at least one element arranged to draw gaseous fluid into the chamber at atmospheric pressure, an exhaust, an ultra-violet source located at one end of the chamber and configured to generate a substantially collimated beam of radiation having a wave length in the range 240 to 290 nm, and an ultra-violet sensor arranged at the other end of the chamber and configured to receive the ultra-violet light emitted by the said ultra-violet source.

Advantageously, the element arranged to draw gaseous fluid into the chamber is a fan.

Preferably, the ultra-violet source is an ultra-violet light emitting diode (UV-LED), and may be solar blind. The output of the ultra-violet source may be adapted to facilitate identification of the said output by the UV sensor, for example by modulation.

The UV sensor and UV source may be driven at a frequency and using known electronic techniques be made phase sensitive, which in the present example would allow the response of the UV sensor due to light from the UV source to be identified over background light.

Advantageously, the instrument comprises at least elements arranged to draw gaseous fluid into the chamber, wherein each element is associated with a filter adapted to remove particulates from gaseous fluid entering the chamber, and wherein one of the elements is associated with a filter adapted to remove ozone from gaseous fluid entering the chamber.

The instrument may further comprise a temperature sensor.

The instrument may further comprise a third element arranged to draw gaseous fluid into the chamber.

Preferably, the instrument comprises a controller, which may include an electronic filter adapted to extract that part of the output signal of the UV sensor corresponding to the input of modulated ultra-violet radiation emitted by the UV source.

Preferably, the controller is programmed with an algorithm which performs the Beer-Lambert Law (C₀₃=ln((I₀)/(I_{c})/σl).

A second aspect of the invention provides a method of determining the concentration of ozone in gaseous fluid using an instrument as described above, comprising the steps of: (i) drawing gaseous fluid through the filter adapted to remove ozone from said gaseous fluid into the chamber to fill said chamber with ozone free gaseous fluid; (ii) powering the ultra-violet source and measuring the output signal of the ultra-violet sensor I₀; (iii) drawing gaseous fluid through the filter adapted to remove particulates from said gaseous fluid into the chamber to fill said chamber with gaseous fluid potentially burdened with ozone; (iv) powering the ultra-violet source and measuring the output signal of the ultra-violet sensor I_{c}; (v) running the algorithm embodied in the controller to establish ozone concentration; and (vi) issuing a signal representative of ozone concentration.

The method may further include the step of measuring the temperature of the gaseous fluid in the chamber and updating a value used in said algorithm which varies with temperature.

Preferably, the method includes the further step of comparing the value of I₀ with threshold values in a range, wherein any value outside the range indicates a fault.

The method may include the further step of operating the element arranged to introduce into the chamber gaseous fluid burdened with a specified concentration of ozone, powering the UV-LED and comparing the output of the UV sensor with the expected output of the UV sensor in the presence of such a concentration of ozone.

The method advantageously comprises the further step of operating the element arranged to introduce into the chamber gaseous fluid burdened with a specified concentration of ozone, and introducing gaseous fluid burdened with a further specified concentration of ozone into the chamber, powering the UV-LED and comparing the output of the UV sensor with expected output of the UV sensor for the further specified concentration of ozone in the chamber.

The method may comprise the further step of issuing an alert signal if the ozone concentration measured by the instrument deviates from the actual concentration by more than a pre-defined amount.

A third aspect of the invention provides a method of controlling the concentration of ozone in a body of gaseous fluid in a controlled environment, comprising the steps of: (i) determining the concentration of ozone in the body of gaseous fluid using an instrument according to the first aspect of the invention by performing the method of the second aspect of the invention; and (ii) increasing the ozone concentration in the body of gaseous fluid by introducing ozone into the body of gaseous fluid.

It is not usually necessary to vent gaseous fluid burdened with ozone from a controlled environment, as ozone has a relatively short half life, meaning that excess ozone decays rapidly. However, where there is a need to control the reduction of ozone concentration in the body of gaseous fluid, for example in the case of malfunction of the ozone generator, gaseous fluid burdened with ozone may be vented from the controlled environment.

A fourth aspect of the invention provides a sterilisation method comprising the steps of: (i) controlling the concentration of ozone in a body of gaseous fluid in a controlled environment according to the method of claim 14; (ii) monitoring and recording the concentration of ozone in a body of gaseous fluid delivered to an object to be sterilised in the controlled environment during a period of sterilization; and (iii) issuing one of two signals at the end of the period of sterilisation, the first signal indicating that ozone above a threshold level of concentration has been issued to the object being sterilised during the period, and the second signal indicating that ozone below a threshold level of concentration has been issued to the object being sterilised during the period.

The instrument of the invention provides a number of advantages. In the ozone detectors of the prior art using mercury discharge lamps, the chamber into which gaseous fluid burdened with air must be introduced is a narrow tube, air being drawn into the tube by a vacuum pump. In the present invention a comparatively large chamber is utilised with fans, rather than vacuum pumps being used to fill the chamber with gaseous fluid. It has been found that deposits can build up on the inside of the walls of the tubes leading to inaccurate measurement. In the present example the chamber is of such dimensions that the chamber walls are not impinged upon by the beam of UV emitted from the UV source. Further, the walls of the chamber fall outside the field of view of the UV detector. This means that if deposits build up on the internal surfaces of the chamber, the accuracy of the detected ozone concentration should not be affected. The size of the chamber also makes the internal surfaces thereof accessible for cleaning Also, the running costs of fans are significantly less than those of vacuum pumps, and the reliability of fans is likely to be better than for vacuum pumps. In the present invention either a solar blind sensor may be used, or a non-solar blind sensor may be used and the output of the UV source modulated such that the element of the output signal of the UV source corresponding to UV light falling thereon from the UV source may be extracted from the said output signal. Hence, the instrument may be fabricated at less cost than instruments of the prior art. Where a non-solar blind sensor is used, UV sensors giving better responses may be used. Since the amounts of ozone to be detected are small, a sensor having a better response may be advantageous. For example, non-solar blind sensors, which are bigger than presently available solar blind sensors, may be used. This allows the distance between the source and the sensor to be increased, which as can be seen from Beer-Lambert equation increases the sensitivity of the measurement.

Various aspects of this invention will become apparent to those skilled in the art from the following detailed description of the preferred embodiment, when read in light of the accompanying drawings.

### BRIEF DESCRIPTION OF THE DRAWINGS

FIG. 1 is a is an exploded view of a part of an instrument according to the invention.

FIG. 2 is a block diagram of a control system of an instrument according to the invention.

FIG. 3 is a schematic representation of the instrument illustrated in FIGS. 1 and 2.

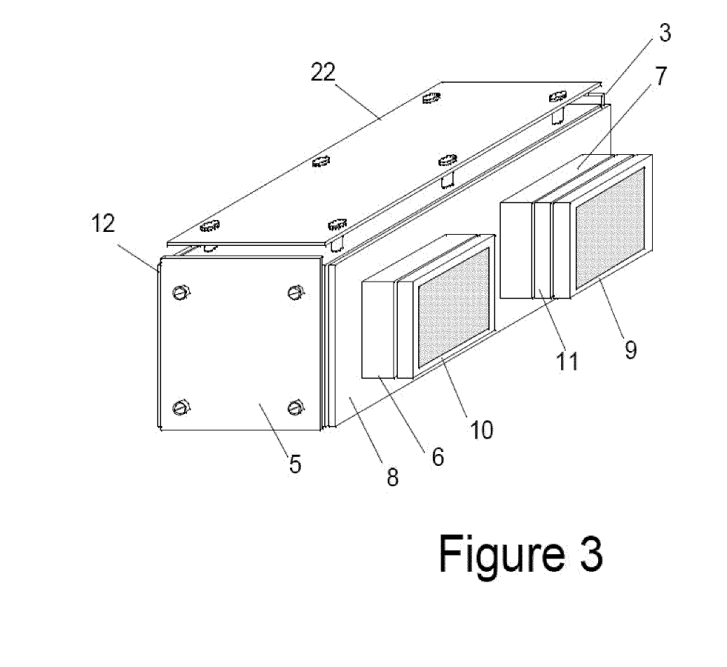

FIG. 4 is a schematic representation of a crop store according to the invention.

### DETAILED DESCRIPTION OF THE PREFERRED EMBODIMENT

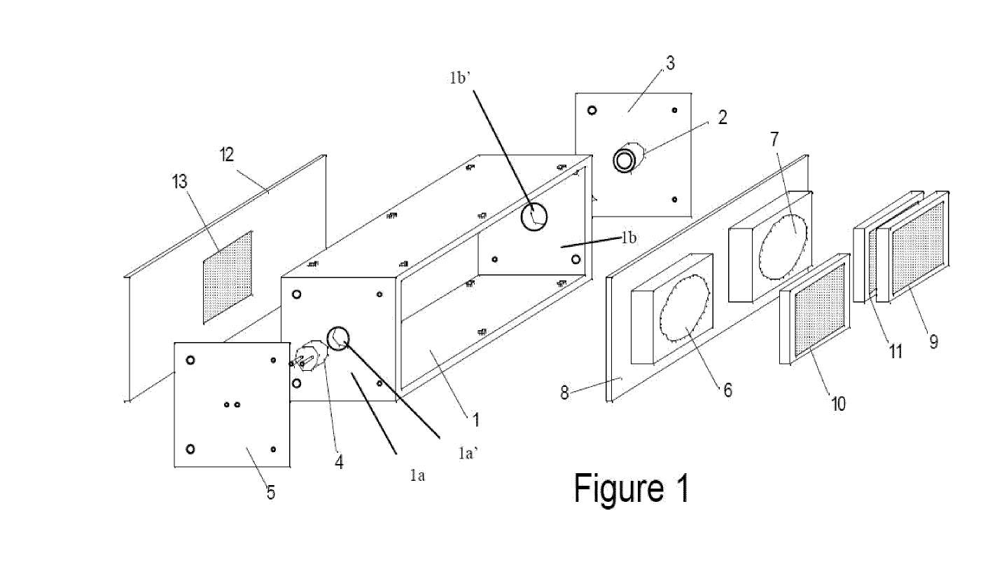

Referring now to FIG. 1, there is illustrated an air sampling apparatus of the instrument according to the invention. The apparatus comprises a chamber in the form of a box made up of an element 1 forming four walls of the box and mounting the other two walls 8 and 12, a UV source 2 and UV sensor 4. The UV source 2 is attached to a circuit board 3, which is itself removably attachable to an end wall 1a of the element 1. Similarly, the UV sensor 4 is attached to a circuit board 5, which is itself removably attachable to the end wall 1b of the element 1.

It is important that the UV source is aligned accurately with the UV sensor 4. To this end, the element 1 may be formed of cast metal such as aluminium. An accurate datum surface may therefore be provided, to which the circuit board 3 on which the UV source is mounted may be attached. Similarly, it is important that an accurate datum surface is provided for the attachment of the circuit board upon which the UV sensor is mounted.

The wall 8 mounts two fans 6, 7. The fans draw air into the chamber through filter media. The filter media 10, through which fan 6 draws air, is configured to remove airborne particles from air being drawn into the chamber by the fan 6.

The fan 7 is associated with filter media 9 and 11. The filter media 9 is configured to remove airborne particles from air drawn into the chamber by fan 7. The filter media 11 aligned in series with filter media 9 is configured to remove ozone from air drawn into the chamber. A suitable filter media would include activated charcoal, manganese-oxide or other ozone absorbing substances.

The wall 12 includes an outlet comprising a perforated plate 13. The outlet 13 is not strictly necessary. Upon activation of a fan to draw gaseous fluid into the chamber through one of the filters any gaseous fluid contained in the chamber is pushed out through the other filter.

Where two fans are provided it is possible that one or other of the fans may always be running However, it may be desirable to switch off the fan in operation for a brief period around the point when the UV signal is being measured, in order to avoid cooling of the LED which might affect the signal emitted thereby.

The UV source 2 comprises an Ultra Violet Light Emitting Diode (UV-LED) and is installed on a printed circuit board (PCB) 3. The UV-LED of the example emits a narrow beam (the edge of the beam being up to 10 degrees either side of the centre axis of the UV-LED) of near-collimated beam of radiation at a wavelength of between 250 nm and 290 nm. One suitable UV-LED emits radiation at a wavelength of 265 nm. In the present example, this is achieved with no optical element beyond the lens of the LED. One type of UV-LED capable of emitting ultra-violet light in the range 250 to 290 nm is an LED based on AlGaN/GaN technology using a metal-oxide vapour deposition process. In the example, the UV-LED includes a ball end which focuses the UV light emitted by the UV-LED.

The UV-LED is mounted on the PCB 3, which comprises electronic circuitry configured to deliver a highly stable current to the UV-LED, the UV-LED emitting optical power of about 300 micro-watts (the UV-LED draws approximately 150 milli-watts of power). The PCB 3 attaches to the end wall 1b in such a manner that the UV-LED projects through an aperture 1b′ and that the beam of radiation emitted from the UV-LED is directed such the UV sensor 4 is illuminated by the beam of radiation. The UV sensor 4 is a solar blind photo-detector, for example a silicon-carbide photo-detector, or a photo-detector based on titanium dioxide semiconductor material. The advantage of using a solar blind photo-detector is that ambient light need not be excluded from the chamber. Of course, if light were excluded from the chamber a photo-detector which is not solar blind may be used.

It is also possible to use a photo-detector which is not intrinsically responsive to the wavelength of UV emitted by the LED by providing a fluorescent element between the photo-detector and the impinging beam. In such a scenario, or where another type of non-solar-blind photo-detector is employed, by modulating the UV-LED (for example by switching the current to the UV-LED on and off by a square wave at a rate of several tens of KHz) and preferably providing a phase sensitive detector, it is possible to recover the signal from the photo-detector that is representative of the light beam traversing the chamber to the exclusion of other sources of ambient radiation by the use of an electronic filter or the like. The electronic filter would typically be a band pass filter with a centre frequency chosen to be the same as the LED modulation frequency.

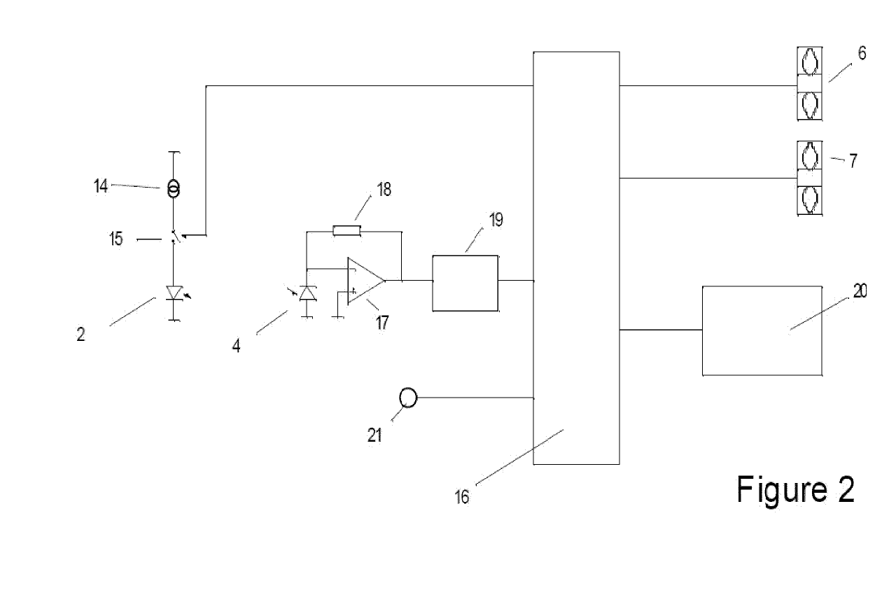

The control system will now be described with reference to FIG. 2. The control system includes a controller 16, which may comprise a microprocessor, a microcontroller, or discrete logic circuits. The controller 16 manages operation of the control system. The controller and its associated components are mounted on a printed circuit board 22.

The controller 16 has a number of inputs, namely: a temperature sensor 21, the output of an analogue to digital converter 19 which itself is connected to the photo-detector via a trans-impedance amplifier arrangement comprising an operational amplifier 17 and a trans-impedance feedback resistor 18. Hence, the controller 16 receives as an input the digital equivalent of signal generated by the photo-detector. In the embodiment described in the invention the analogue to digital converter has a resolution of at least sixteen binary bits in order to resolve the lowest concentrations of ozone to be measured.

The purpose of the temperature sensor is to allow the controller 16 to make adjustments to the recorded value of ozone concentration to compensate for air density variation with temperature. In the example, the temperature sensor 21 is connected to an analogue input of the controller 16.

The fans 6 and 7 are connected to controller output ports in order that they may be switched on and off. Where an additional fan is provided this would also be connected to a controller output port. Similarly, if the aperture of the plate 13 is provided with a closing means, an actuator controlling opening and closing thereof would be connected to an output port of the controller.

As can be seen from FIG. 2, the UV-LED 2 is powered by a current source 14, which is switched on and off by a switch 15, which is commanded by the controller 16.

A user interface 20 is connected to the controller 16. The user interface may include a visual display unit and/or annunciation means, such as a screen and/or a speaker which allow a user to be informed of ozone concentration levels, instrument malfunction, etc., and/or a keypad to allow a user to input information into the controller 16, or retrieve information generated by the controller.

Where the instrument is configured as part of a control system for an environment in which the concentration level of ozone must be controlled and maintained and/or adjusted, the controller 16 may be programmed with an ozone concentration cycle. For example, in a crop store it may be desirable to increase the concentration of ozone at night, for the better preservation of the stored crops, yet in the day time, when people are working in the store it may be necessary to reduce the ozone concentration. The controller 16 may be connected to an apparatus for controlling the supply of ozone, so that when the measured concentration of ozone is below the desired concentration, additional ozone may be introduced. Similarly, the controller may be connected to apparatus for controlling the ventilation system of the store. If the ozone concentration is above a desired concentration, the ozone generator may be switched off and/or a ventilation system may be operated to allow air burdened with ozone to pass from the store.

For example, if the detected ozone concentration during working hours were above a threshold amount, e.g. 80 ppb, then the ozone generator would be switched off and/or a ventilation system activated.

It is desirable that the instrument may measure ozone concentration in air down to as little as 10 parts per billion and up to as much as 10 parts per million by volume. In many countries where there are laws relating to the maximum concentration of ozone in air, the limit is often set at 80 ppb, whereas in the UK it is 200 ppb.

The apparatus functions by first switching on fan 7. This purges the chamber 1. The fan 7 is left running to fill the chamber 1 with clean and ozone free air. At this point the chamber 1 is filled with air free of ozone. In the example the fan 7 is left running during the following step, or at least a part thereof.

The UV-LED is then powered up and left for a short period until its output has stabilised. A measurement of beam strength, represented by the output current (I₀) of the photo-detector, is then taken. The fan 7 may be switched off after the output of the UV-LED has stabilised but before the measurement of beam strength is taken. As mentioned above, switching off the fan 7 can give a more accurate measurement as any cooling effect of the fan on the UV-LED is removed.

The fan 7 is then switched off and the fan 6 switched on. The fan 6 runs, filling the chamber with air depleted of particulates but not ozone.

The UV-LED is then powered up and a measurement of beam strength, represented by the output current (I_{c}) of the photo-detector, is taken. The fan 6 may be switched off after the output of the UV-LED has stabilised but before the measurement of beam strength is taken. As mentioned above, switching off the fan 6 can give a more accurate measurement as any cooling effect of the fan on the UV-LED is removed.

The output current (I_{c}) will be less than (I₀) where ozone is present since ozone absorbs some of the ultra-violet light emitted by the UV-LED.

The cycle is repeated as often as is necessary for the monitoring or control purpose for which the instrument is deployed.

From the values of (I₀) and (I_{c}) and other fixed parameters, namely the co-efficient of absorption of ozone and the distance between the UV source and the UV detector, the concentration of ozone in the air sample may be determined. This is done using the well known Beer-Lambert Law which is described in the equation:

C₀₃=ln(I₀)/(I_{c})/σl

Where C₀₃ is the required concentration of ozone; σ is the absorption co-efficient for ozone at the UV-LED's emission wavelength, at conditions of standard air temperature and pressure; l is the distance across the chamber between the UV-LED and the photo-detector; I₀ is the measurement recorded in the chamber with ozone removed, and I_{c} is the measurement recorded in the chamber without ozone having been removed.

In the illustrated embodiment l is equal to 0.1 m, and σ is equal to 308 atm⁻¹ cm⁻¹.

The outlet 13 may be omitted. In such a case gaseous fluid occupying the chamber exits through the filter whose associated fan is switched off. The chamber is filled with air by the respective fans and the UV-LED is powered up and the output from the photo-detector taken with the fans blowing air, which is either burdened with ozone or not, depending on which fan is actuated. A measurement routine where the outlet 13 is omitted is described below:

a) The current source (14) is enabled to feed the UV-LED (2) by means of electronic switch (15), followed by a short settling period to allow the UV-LED's emission to stabilize. The current source 14 must be substantially stable in order to provide adequate resolution for the instrument. The current source 14 may include an XFET (eXtra implanted junction FET) device to provide high accuracy and low temperature drift performance. Such a device is available from Analog Devices, Inc., and uses temperature drift curvature correction technology to minimize voltage change vs. temperature nonlinearity. Such an XFET allows operation of the instrument at much lower supply headroom voltages than the more usual buried Zener references, which may be important in this application where the UV-LED of the example has a high forward voltage requirement for an LED device. However, Zener and/or other reference devices may be used.

b) The controller (16) next enables fan (7) whilst disabling fan (6), so that air, purged of any ozone present, is forced through the chamber. After a short period to allow the chamber to be purged, the controller (16) instructs the analogue to digital converter (19) to measure the output from the transimpedance amplifier (17). The electronic controller (16) receives and stores this value in its memory. This value is designated as the I₀ value for entry into the Beer-Lambert equation.

c) Before proceeding, the controller (16) next assesses whether the I₀ value is within acceptable limits. If it is not the controller signals a fault condition by issuing an appropriate message to a human operator through the annunciation means (20). If I₀ is within acceptable limits, the controller proceeds to the next step.

d) In the next step the fan (7) is switched off, fan (6) is switched on and, after a suitable interval to purge the chamber, the electronic controller (10) instructs the analogue to digital converter (19) to measure the output from the transimpedance amplifier (17). The electronic controller (16) receives and stores this value in its memory. This value is designated as the I_{c} value for entry into the Beer-Lambert equation.

e) Optionally, the controller (16) may perform a measurement of ambient temperature by way of the temperature sensor (21), and store this in memory in readiness for applying a correction to the calculation to be made below under step f).

f) The controller 16 next performs a calculation according to the Beer-Lambert equation, utilising the I₀ and I_{c} values so obtained, together with the appropriate constants, σ and l. In the embodiment described, l is equal to 0.1 m, and σ is equal to 308 atm⁻¹ cm⁻¹. Given these constants, a difference of −0.003% between I₀ and I_{c} will be calculated in an environment bearing a concentration of 10 parts per billion by volume (ppbv) ozone. The value of ozone concentration so calculated may be presented by means of an appropriate message to a human operator through the input and annunciation means (20). The controller 16 may also be used to provide an external stimulus, for example to ozone generating equipment, when it is above, or below, certain limits of ozone concentration as set by an operator through the input and annunciation means (20).

g) Finally, the controller instructs the UV-LED (2) to be turned off by means of the electronic switch (15), pending the next measurement cycle.

This measurement cycle as described in a) to g) repeats at a frequency appropriate to the application. A reduced frequency will allow considerable power saving in applications where permanent electrical supply is limited. However, in applications requiring a faster response, this can be accommodated.

FIG. 2 illustrates a control system of the instrument of the invention. The control system controls activation of the fans 6, 7, and the switching on and off of the UV-LED. The control system includes a micro-processor which is programmed to perform the calculation of ozone concentration according to the Beer-Lambert law.

The control system provides communication to a user interface 20, for the annunciation of messages, and external interfacing with other devices to enable the concentration of ozone to be utilised by an operator and/or to directly control ancillary equipment including ozone generation apparatus.

For example, the instrument of the invention may be deployed simply for monitoring the concentration of ozone in an environment, or alternatively, the instrument may form part of a control system for an environment where the concentration of ozone is controlled, such as a crop store.

The control system may be configured to alert a human operator if the ozone concentration indicated is outside certain limits (such limits may indicate that the UV-LED or the UV sensor is coming to the end of its life).

In another embodiment, the instrument may form part of a sterilisation apparatus, in which ozone is the sterilisation agent, and to verify that sterilisation has taken place systems must be capable of monitoring and recording ozone concentration over a period of time. To verify that sterilisation has taken place it must be possible to show that the object being sterilised has been subject to ozone in a concentration above a threshold level for a certain period of time. By executing the measuring cycle of the instrument such verification information may be gathered.

The instrument may include a further fan arranged to introduce into the chamber air burdened with a specified concentration of ozone. The UV-LED is powered up and the output of the UV sensor is compared with the expected output of the UV sensor in the presence of such a concentration of ozone.

Further, the control system may, from time to time, switch off all fans other than the fan arranged to introduce into the chamber air burdened with a specified concentration of ozone, and introduce air burdened with a further specified concentration of ozone into the chamber. The UV-LED is powered up and the output of the UV sensor is compared with expected output of the UV sensor for the further specified concentration of ozone in the chamber. If the concentration measured by the instrument deviates from the actual concentration by more than a pre-defined amount, an alert signal is issued by the control system.

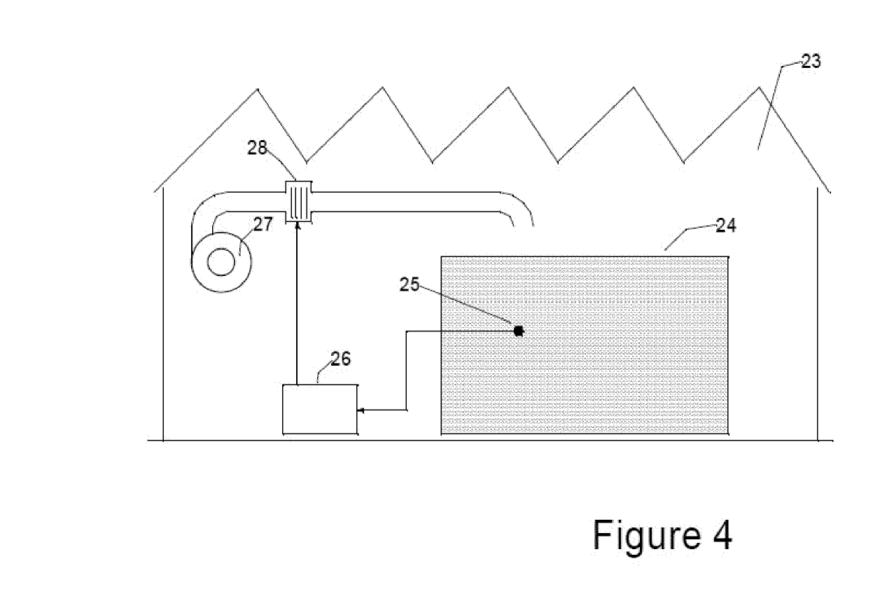

FIG. 4 schematically illustrates the instrument 26 incorporated in a crop store 23 loaded with harvested crops 24. A sample tube 25 located in a suitable position within the body of crops conveys a sample of air to the instrument 26. The instrument controls the on or off state of an ozone generator 28 that is incorporated within a ventilation and cooling system, 27.

The principle and mode of operation of this invention have been explained and illustrated in its preferred embodiment. However, it must be understood that this invention may be practiced otherwise than as specifically explained and illustrated without departing from its spirit or scope.

What is claimed is:

1. An instrument for determining ozone concentration in a gaseous fluid comprising a chamber, at least one filter adapted to remove particulates from gaseous fluid entering the chamber, and at least one filter adapted to remove ozone from gaseous fluid entering the chamber, at least one element arranged to draw gaseous fluid into the chamber at atmospheric pressure, an exhaust, an ultra-violet source located at one end of the chamber and configured to generate a substantially collimated beam of radiation having a wave length in the range 240 to 290 nm, and an ultra-violet sensor arranged at the other end of the chamber and configured to receive the ultra-violet light emitted by the said ultra-violet source.

2. An instrument according to claim 1, wherein the ultra-violet source is an ultra-violet light emitting diode (UV-LED).

3. An instrument according to claim 1, wherein the instrument includes a circuit adapted to drive the UV-LED with a substantially stable current.

4. An instrument according to claim 1, wherein the ultra-violet sensor is solar blind.

5. An instrument according to claim 1, wherein the optical output of the ultra-violet source is adapted to facilitate identification of the said output by the UV sensor.

6. An instrument according to claim 5, wherein the form of adaptation of the output of the ultra-violet source is by modulation of the electrical input thereof.

7. An instrument according to claim 1, comprising at least two elements arranged to draw gaseous fluid into the chamber, wherein each element is associated with a filter adapted to remove particulates from gaseous fluid entering the chamber, and wherein one of the elements is associated with a filter adapted to remove ozone from gaseous fluid entering the chamber.

8. An instrument according to claim 1, further comprising a temperature sensor.

9. An instrument according to claim 7, comprising a third element arranged to draw gaseous fluid into the chamber.

10. An instrument according to claim 1, wherein the at least one element arranged to draw gaseous fluid into the chamber is a fan.

11. An instrument according to claim 1, further comprising a controller.

12. An instrument according to claim 11, wherein the controller includes an electronic filter adapted to extract that part of the output signal of the UV sensor corresponding to the input of modulated ultra-violet radiation emitted by the UV source.

13. An instrument according to claim 11, wherein the controller is programmed with an algorithm which performs the Beer-Lambert Law (C₀₃=ln(I₀)/ (I_{c})/σl).

14. A method of controlling the concentration of ozone in a body of gaseous fluid in a controlled environment, comprising the steps of:
a) determining the concentration of ozone in the body of gaseous fluid using an instrument for determining ozone concentration in a gaseous fluid comprising a chamber, at least one filter adapted to remove particulates from gaseous fluid entering the chamber, and at least one filter adapted to remove ozone from gaseous fluid entering the chamber, at least one element arranged to draw gaseous fluid into the chamber at atmospheric pressure, an exhaust, an ultra-violet source located at one end of the chamber and configured to generate a substantially collimated beam of radiation having a wave length in the range 240 to 290 nm, and an ultra-violet sensor arranged at the other end of the chamber and configured to receive the ultra-violet light emitted by the said ultra-violet source, by performing the method steps of:
i) drawing gaseous fluid through the filter adapted to remove ozone from said gaseous fluid into the chamber to fill said chamber with ozone free gaseous fluid;
ii) powering the ultra-violet source and measuring the output signal of the ultra-violet sensor I₀;
iii) drawing gaseous fluid through the filter adapted to remove particulates from said gaseous fluid into the chamber to fill said chamber with gaseous fluid potentially burdened with ozone;
iv) powering the ultra-violet source and measuring the output signal of the ultra-violet sensor I_{c};
v) running the algorithm embodied in the controller to establish ozone concentration;
vi) issuing a signal representative of ozone concentration; and
b) increasing the ozone concentration in the body of gaseous fluid by introducing ozone into the body of gaseous fluid, and/or reducing the concentration of ozone in the body of gaseous fluid by venting gaseous fluid burdened with ozone from the controlled environment.

15. A method according to claim 14, including the step of measuring the temperature of the gaseous fluid in the chamber and updating a value used in said algorithm which varies with temperature.

16. A method according to claim 14, including the further step of comparing the value of I₀ with threshold values in a range, wherein any value outside the range indicates a fault.

17. A method according to claim 14, including the further step of operating the element arranged to introduce into the chamber gaseous fluid burdened with a specified concentration of ozone, powering the UV-LED and comparing the output of the UV sensor with the expected output of the UV sensor in the presence of such a concentration of ozone.

18. A method according to claim 17, comprising the further step of operating the element arranged to introduce into the chamber gaseous fluid burdened with a specified concentration of ozone, and introducing gaseous fluid burdened with a further specified concentration of ozone into the chamber, powering the UV-LED and comparing the output of the UV sensor with expected output of the UV sensor for the further specified concentration of ozone in the chamber.

19. A method according to claim 18, comprising the further step of issuing an alert signal if the ozone concentration measured by the instrument deviates from the actual concentration by more than a pre-defined amount.

20. A sterilisation method comprising the steps of:
i. controlling the concentration of ozone in a body of gaseous fluid in a controlled environment according to the method of claim 14;
ii. monitoring and recording the concentration of ozone in a body of gaseous fluid delivered to an object to be sterilised in the controlled environment during a period of sterilization; and
issuing one of two signals at the end of the period of sterilisation, the first signal indicating that ozone above a threshold level of concentration has been issued to the object being sterilised during the period, and the second signal indicating that ozone below a threshold level of concentration has been issued to the object being sterilised during the period.
